(19) United States
(12) Patent Application Publication
(10) Pub. No.: US 2023/0031433 A1
(43) Pub. Date: Feb. 2, 2023

(54) COMPOSITIONS AND METHODS FOR MANAGING INFECTIONS OF A URINARY TRACT

(71) Applicant: METAGENICS, INC, Aliso Viejo, CA (US)

(72) Inventors: An-Katrien VYNCKIER, Bruges (BE); Mieke VAN DEN DRIESSCHE, Oud-Heverlee (BE)

(21) Appl. No.: 17/784,157

(22) PCT Filed: Dec. 18, 2020
(86) PCT No.: PCT/US2020/066064

§ 371 Date: Jun. 10, 2022

Related U.S. Application Data
(60) Provisional application No. 62/950,553, filed on Dec. 19, 2019.

(51) Int. Cl. A23L 33/125; A61K 31/7004; A61K 31/702; A61K 31/352; A61K 31/353; A61K 31/375; A61K 36/82; A61P 13/02; A61K 31/19; A61K 36/537; A61P 31/00; A23L 33/105; A23L 33/15

(52) U.S. Cl. CPC A23L 33/125; A61K 31/7004; A61K 31/702; A61K 31/352; A61K 31/353; A61K 31/375; A61K 36/82; A61P 13/02; A61K 31/19; A61K 36/537; A61P 31/00; A23L 33/105; A23L 33/15; A23V 2002/00

(57) ABSTRACT

A composition for treating, ameliorating, or preventing an infection in a mammalian urinary tract is provided. The composition comprises a monosaccharide comprising a D-mannose, an oligosaccharide comprising an isolated human milk oligosaccharide (HMO), a flavonoid comprising an A-type proanthocyanidin, quercetin, a vitamin comprising ascorbic acid, a green tea extract, and optionally a triterpenoid comprising ursolic acid. A method of treating, ameliorating, or preventing an infection in a urinary tract of a mammal is also provided, and comprises administering the composition to the mammal.

### CROSS-REFERENCE TO RELATED APPLICATIONS

This application claims the benefit of U.S. Provisional Application 62/950,553, filed Dec. 19, 2019, the disclosure of which is incorporated by reference in its entirety.

### TECHNICAL FIELD

The present disclosure generally relates to a composition for treating, ameliorating, or preventing an infection in a mammalian urinary tract and, more specifically, to a composition comprising at least a D-mannose and an isolated human milk oligosaccharide (HMO) and to related uses and methods associated with the composition.

### BACKGROUND

Urinary tract infections (UTIs), which are defined microbiologically as the inflammatory response of the urothelium to microbial pathogens, are among the most common bacterial infections affecting an estimated 150 million people each year worldwide. The true incidence is difficult to accurately define (owing to differences in reporting methods and diagnostic criteria), but half of all women have been estimated to experience a UTI in their lifetime, and up to 50% of these will have recurrent infection within the following 6-month period. Observational studies have found an average recurrence rate in women of 2.6 infections per year. The incidence of UTI in men is considerably lower than that in women, with an estimated lifetime prevalence of 13.7%. In 2007, UTI recurrence accounted for 10.5 million outpatient consultations and 2-3 million emergency department visits in the USA alone. In addition, UTIs are the most common cause of infection in hospitalized patients, accounting for 17.2% of all nosocomial infections in England. Furthermore, UTIs result in considerable patient morbidity and time off work; hence, the management of this condition incurs large financial costs, estimated at $3.5 billion in the USA per year.

Urinary tract infections are the result of an interaction between bacterial virulence and host defense factors that compete to invade or protect the host, respectively. An important side-note is that the vast majority of urinary health research has been conducted without knowledge or consideration of the female urinary microbiota (FUM), which are communities of microbes present in the lower urinary tract of most adult women. Prior to the discovery of the FUM in 2012, clinicians relied on the assumption of bladder sterility and depended on the standard urine culture to be the ‘gold-standard’ for detection of clinically relevant urinary organisms. They can no longer depend on either. Given the existence of the female urinary microbiota, the simple presence of bacteria in the lower urinary tract should not be taken as evidence of infection.

Management of women with frequent symptomatic UTI can be particularly vexing for both patients and their treating physicians. For the patient, each UTI recurrence is associated with days of lower urinary tract symptoms, general malaise, and restrictions on everyday activities. For physicians, an etiology is often never elucidated, making patient counseling difficult. Additionally, current prophylactic measures are limited, often ineffective, and may be associated with untoward side effects.

Currently, the most effective treatment for recurrent urinary tract infections in women is antibiotics. However, the limitation for this treatment is the duration and dosage of antibiotics and the resistance that bacteria develop after a long period of administration. To this end, prolonged antibiotic use in women has resulted in the emergence of resistant organisms in their urine.

Accordingly, it is desirable to provide an improved composition for treating, ameliorating, or preventing an infection in a mammalian urinary tract and methods relating to the same. Furthermore, other desirable features and characteristics will become apparent from the subsequent summary and detailed description and the appended claims, taken in conjunction with the foregoing technical field and background.

### SUMMARY OF THE DISCLOSURE

A composition is provided. The composition comprises a monosaccharide comprising a D-mannose, an oligosaccharide comprising an isolated human milk oligosaccharide (HMO), a flavonoid comprising an A-type proanthocyanidin, quercetin, a vitamin comprising ascorbic acid, a green tea extract, and optionally a triterpenoid comprising ursolic acid.

A method of treating, ameliorating, or preventing an infection in a urinary tract of a mammal is also provided, and comprises administering the composition to the mammal.

### DETAILED DESCRIPTION

A composition for treating, ameliorating, or preventing an infection in a mammalian urinary tract is provided herein. The composition comprises a monosaccharide comprising a D-mannose, an oligosaccharide comprising an isolated human milk oligosaccharide (HMO), flavonoids comprising an A-type proanthocyanidin and quercetin, a vitamin comprising ascorbic acid, a green tea extract, and optionally a triterpenoid comprising ursolic acid, which are each described in turn below. A method of treating, ameliorating, or preventing an infection in a urinary tract of a mammal, comprising administering the composition to the mammal, is also provided. A foodstuff or beverage comprising the composition is also provided, and can be used in the methods described herein. A combination for use in a method of treating, ameliorating, or preventing an infection in a mammalian urinary tract with the combination comprising a D-mannose and an isolated human milk oligosaccharide (HMO) is also provided. Without being bound by theory, it is believed that the combination of the D-mannose and the isolated HMO, as well as the A-type proanthocyanidin, quercetin, ursolic acid, green tea extract, and/or ascorbic acid in some instances, is synergistic as a prophylactic and therapeutic strategy for reducing frequency and severity of urinary tract infections in mammals.

As used herein, the singular forms “a,” “an,” and “the” are meant to include plural referents unless the context clearly dictates otherwise.

As used herein, an “embodiment” means that a particular feature, structure, or characteristic is included in at least one or more manifestations, examples, or implementations of this disclosure. Furthermore, the particular features, structures, or characteristics may be combined in any suitable manner, as would be apparent to a person skilled in the art. Combinations of features of different embodiments are all meant to be within the scope of the disclosure, without the need for explicitly describing every possible permutation by example. Thus, any of the described and/or claimed embodiments can be used in any combination.

As used herein, the term “weight percent” (and thus the associated abbreviation “wt. %”) typically refers to a percent by weight expressed in terms of a weight of dry matter. As such, it is to be appreciated that a wt. % can be calculated on a basis of a total weight of a composition, or calculated from a ratio between two or more components/parts of a mixture (e.g. a total weight of dry matter).

As used herein, the terms “about” and “approximately,” when referring to a specified, measurable value (such as a parameter, an amount, a temporal duration, and the like), is meant to encompass the specified value and variations of and from the specified value, such as variations of +/−10% or less, optionally+/−5% or less, optionally+/−1% or less, optionally+/−0.1% or less of and from the specified value, insofar as such variations are appropriate to perform in the disclosed embodiments. Thus the value to which the modifier “about” or “approximately” refers is itself also specifically disclosed.

As used herein, the term “animal” refers to an organism of the kingdom Animalia that has a urinary tract. Examples of animals include mammals (i.e., vertebrates of the class Mammalia). All mammals have urinary tracts. Mammals that are specifically contemplated herein are domesticated mammals, such as dogs, cats, goats, sheep, pigs, cattle, horses, donkeys, camels, and the like. Additional mammals that are specifically contemplated herein include semi-domesticated mammals and mammals that are routinely bred in captivity. Of course, the term mammal also encompasses humans (which may be referred to as “people” and/or “person(s).” When describing a human, the term “adult” is typically used herein to refer to a human that has reached sexual maturity. By contrast, the terms “child” and “juvenile” are used herein to refer to a human that has not yet reached sexual maturity. Typically, the term “child” means a human subject between the stage of birth and the age of about 10 (i.e., childhood), and the term “juvenile” means a human subject that is greater than the age of about 10 and who has not completed the stage of puberty. Of course, the terms child, juvenile, adult, and infant are all encompassed by the term human, which is itself a subcategory of mammal, which is a subcategory of animal as defined herein.

As used herein, the terms “treatment” and “treat” refer to and encompass prophylactic (i.e., preventive), modifying, and curative treatments. As such, these terms including treatment of patients (e.g. humans) at risk of contracting a disease or suspected to have contracted a disease, as well as patients who are ill or have been diagnosed as suffering from a disease or medical condition.

As used herein, the term “therapeutically effective amount” relates to an amount (i.e., a quantity) of a composition (e.g. the prebiotic composition of the present embodiments) required to achieve a particular therapeutic and/or prophylactic effect, such as in treating a patient. Likewise, as used herein, the term “physiologically effective amount” relates to an amount of a composition required to achieve a desired physiological effect. As will be understood by one of skill in the art, such effective amounts are typically measured and/or expressed in terms of grams per day (g/day), or a derivative thereof (e.g. milligrams per day (mg/day)).

As used herein, the term “synergism” or “synergy” refers to an effect in which two or more agents (e.g. compounds) working together to produce a result not obtainable by any of the agents independently. As such, the term “synergistic amount” refers to amounts of the two or more agents at which a more pronounced (e.g. greater) effect is achieved as compared to the effects of each agent alone.

As used herein, the term “foodstuff” refers to a material that may be used as a food. As such, in certain instances the term foodstuff is used to describe a composition that may be consumed (e.g. by eating) by a living organism (e.g. a mammal), such as for nourishment and/or sustenance.

As used herein, the term “beverage” refers to a potable liquid or other non-solid composition. As such, in certain instances the term beverage is used to describe a non-solid (e.g. liquid, slurry, suspension, etc.) composition that may be consumed by a living organism for nourishment and/or sustenance. As such, in particular instances the terms “beverage” and “foodstuff” may overlap. In certain instances, the term “nutritional composition” is used to describe a foodstuff and/or beverage formulation that can be eaten or drunk by a human subject for nutrition.

As used herein, the term “functional food additive” refers to an ingredient, additive, component, or supplement suitable for incorporation in a foodstuff and/or beverage to confer a technical, nutritional, and/or health benefit (i.e., a function) to a host that consumes the foodstuff and/or beverage. The “functional food additive” can be added to different types of food including, but not limited to, medical foods, dietetic foods, and supplements.

As used herein, the term “medical food” is typically used to refer to a food for a special dietary use, such as a food formulated for dietary management of a medical condition (e.g. based upon scientific or medical evaluation). However, it is to be appreciated that the term “medical food” may have one or more particular definitions depending on, for example, geographic location, specific use, regulatory agency, and the like. For example, in certain cases, the term medical food may be defined as a food which is formulated to be consumed or administered enterally under the supervision of a physician and which is intended for the specific dietary management of a disease or condition for which distinctive nutritional requirements, based on recognized scientific principles, are established by medical evaluation (see, e.g. section 5(b) of the U.S. Orphan Drug Act (21 U.S.C. 360ee (b) (3)), which is incorporated herein by reference). In these or other instances, the term medical food may be defined as a food for special dietary use as a food that has been specially processed or formulated to meet the particular requirements of a person: (a) in whom a physical or physiological condition exists as a result of a disease, disorder, or injury; or (b) for whom a particular effect, including but not limited to weight loss, is to be obtained by a controlled intake of food (see, e.g. section B.24.001 of the Canadian Food and Drug Regulations (FDR, C.R.C., c. 870)(as amended 13 Jun. 2017)), which is incorporated herein by reference).

As used herein, the term “supplement” relates to a nutritional supplement which is a concentrated source of nutrient or alternatively other substances with a nutritional or physiological effect whose purpose is to supplement the normal diet.

As used herein, the term “peptide” describes a molecule having 2 or more amino acids joined together by a peptide bond. As will be understood by those of skill in the art, the term “peptide” encompasses oligopeptides (i.e., peptides comprising 20 or fewer, optionally 10 or fewer amino acids, e.g. di-, tri-, tetra-, and pentapeptides, etc.) polypeptides (i.e., peptides comprising greater than 10, optionally greater than 20 amino acids), proteins (i.e., organic compounds comprising amino acids linked via peptide bonds in a linear chain and folded into a globular form), enzymes (i.e., functional proteins), and the like. Such peptides may include any known amino acid, such as the 20 gene-encoded amino acids or others such as selenocysteine. Additionally, the term “peptide” also encompasses naturally modified peptides, e.g. peptides naturally and/or synthetically modified by glycosylation, acetylation, phosphorylation, and the like, or any combination thereof. Furthermore, the term “peptide” also encompasses branched peptides, especially those known to have therapeutic or beneficial effects. It is to be appreciated that the peptides described herein may be produced recombinantly, synthetically, or semi-synthetically, or obtained from natural sources (e.g. via isolation after hydrolysis of a protein and/or enzyme).

As used herein, the term “lipid” refers to naturally occurring and/or synthetic small molecules that exhibit hydrophobic or amphiphilic properties and typically form vesicles, multilamellar/unilamellar liposomes, and/or membranes in an aqueous environment. As will be understood by one of skill in the art, the term “lipid” encompasses oils, fats (e.g. triglycerides), fatty acids, waxes, sterols, fat-soluble vitamins (e.g. vitamin A, D, E, K, etc.), glycerolipids (e.g. monoglycerides, diglycerides, triglycerides, glycerols, etc.), phospholipids, Sphingolipids, sterols, prenols, saccharolipids, and the like.

As introduced above, the composition comprises a monosaccharide component, which comprises, alternatively consists essentially of, alternatively consists of a D-mannose. Examples of suitable monosaccharides for inclusion in the monosaccharide component (e.g. when comprising the D-mannose) include, but are not limited to, glucose, dextrose, fructose, galactose, mannose (i.e., isomers, etc., other than D-mannose), ribose, deoxyribose, and the like. Such monosaccharides may be used the form of the corresponding D-isomer, L-isomer, or a combination thereof. The monosaccharide component may be present in an amount of from 100 mg to 4,000 mg, alternatively from 1,000 mg to 3,000 mg, alternatively from 1,600 mg to 2,400 mg, each based on a total weight of a serving of the composition. In various embodiments, the serving of the composition is adapted (e.g. in form and/or size) to be consumed by a human selected from the group of adults, juveniles, and a combination thereof. In certain embodiments, the serving is not adapted to be consumed by an infant.

As introduced above, the composition comprises the D-mannose (i.e., as the monosaccharide component, or a component thereof). Virulence factors of enteric bacteria, such as E. coli, include type 1 fimbriae or pili, which are proteinaceous appendages extending from the bacterial cell and are integral for attachment. The type 1 pili are composed of Fim proteins with a FimH adhesin molecule at the tip of the fibrillum. During bacterial colonization, the FimH adhesins may bind to carbohydrate-containing glycoprotein receptors on the epithelium of the urinary tract. Without being bound by theory, it is believed that the D-mannose may inhibit bacterial adherence to urothelial cells, e.g. by binding and blocking FimH adhesion. In some instances, it is believed that the D-mannose may act as a competitive inhibitor of bacterial adherence, as it may be similar in structure to the binding site of urothelial glycoprotein receptors.

In certain embodiments, the D-mannose comprises α-D-mannose. Without being bound by theory, it is believed that the anti-adhesive effect of mannose depends on the configuration of the molecule. To this end, the D-isomer and α-anomer (i.e., α-D-mannose) can bind and block the FimH adhesion. The D-mannose may be present in an amount of at least 50 wt. %, alternatively at least 75 wt. %, alternatively at least 90 wt. % based on a total weight of the monosaccharide component. In these or other embodiments, the composition may comprise any amount of the D-mannose, such as from 5 to 99, alternatively from 50 to 95, alternatively from 70 to 90, wt. % of the D-mannose, based on a total weight of the composition. It is to be appreciated that the particular amount of the D-mannose present in the composition may vary in accordance with the total weight of the composition, which may be measured and/or described on the bases of a sample, a serving size, a batch, or an average of one or more of such samples, serving sizes, or batches. Typically, the amount of the D-mannose present in the composition is measured and/or described with reference to a serving size of the composition as described herein.

As introduced above, the composition comprises an oligosaccharide component, which comprises, alternatively consists essentially of, alternatively consists of, an isolated human milk oligosaccharide (HMO). Examples of suitable oligosaccharides for inclusion in the oligosaccharide component include, but are not limited to, fructo-oligosaccharides, galacto-oligosaccharides, mannan-oligosaccharides, isomalto-oligosaccharide, human milk oligosaccharides (HMO) (i.e., different and in addition to the required HMO described herein), and the like. The oligosaccharide component may be present in an amount of from 100 mg to 1,000 mg, alternatively from 100 mg to 400 mg, alternatively from 200 mg to 300 mg, each based on a total weight of a serving of the composition. In various embodiments, the serving is adapted to be consumed by a human selected from the group of adults, juveniles, and a combination thereof. In certain embodiments, the serving is not adapted to be consumed by an infant.

As introduced above, the oligosaccharide component comprises the isolated human milk oligosaccharide (HMO). The term “isolated” as utilized herein means that the HMO is not provided to the composition directly from a naturally-occurring source, such as from milk, including human breast milk, but instead may be synthesized to form the HMO, derived from naturally occurring precursors, or derived from a naturally-occurring source so long as the HMO is isolated from other components of the naturally-occurring source that are not the HMO. Without being bound by theory, it is believed that the isolated HMO may reduce microbial infections by serving as antiadhesive antimicrobials. As introduced above, many viral, bacterial or protozoan pathogens need to adhere to mucosal surfaces to colonize or invade a host and cause disease. Pathogen adhesion is often initiated by lectin—glycan interactions. For example, Escherichia coli with type 1 fimbriae bind to mannose-containing glycans, whereas E. coli with S fimbriae as well as Helicobacter pylori bind to sialylated glycans. Glycan-mediated attachment mechanisms have also been described for many viruses like noroviruses or rotaviruses. The isolated HMO may resemble mucosal cell surface glycans thereby serve as soluble decoy receptors to prevent pathogen binding and reduce the risk of infections.

The isolated HMO may be selected from the group of 2′-O-fucosyllactose (2′-FL), 3-fucosyllactose (3-FL), 3′-O-sialyllactose (3 ‘-SL), 6′-O-sialyllactose (6’-SL), lacto-N-tetraose (LNT), lacto-N-neotetraose (LNnT), lacto-N-hexaose (LNH), iso-lacto-N-octaose, iso-lacto-N-neooctaose, para-lacto-N-octaose, lacto-N-fucopentaose I (LNFP I), lacto-N-fucopentaose II (LNFP II), lacto-N-fucopentaose III (LNFP III), lacto-N-fucopentaose V (LNFP V), LS-tetrasaccharide a (LST a), LS-tetrasaccharide b (LST b), LS-tetrasaccharide c (LST c), disialylliacto-N-tetraose (DSLNT), difucosyllactose (DFL), and combinations thereof.

In certain embodiments, the isolated HMO comprises 2′-O-fucosyllactose (2′-FL), 3′-0-sialyllactose (3′-SL), or a combination thereof. Without being bound by theory, it is believed that sialic acid-containing fractions of isolated HMO (e.g. 3′-SL) reduce E. coli-mediated MAPK and NF-κB activation. The isolated HMO may be present in the oligosaccharide component in an amount of at least 50 wt. %, alternatively at least 75 wt. %, alternatively at least 90 wt. %, each based on a total weight of the oligosaccharide component. In these or other embodiments, the composition may comprise any amount of the isolated HMO, such as from 1 to 90, alternatively from 2 to 30, alternatively from 5 to 15, wt. % of the isolated HMO, based on a total weight of the composition. It is to be appreciated that the particular amount of the isolated HMO present in the composition may vary in accordance with the total weight of the composition, which may be measured and/or described on the bases of a sample, a serving size, a batch, or an average of one or more samples, serving sizes, or batches. Typically, the amount of the isolated HMO present in the composition is measured and/or described with reference to a serving size of the composition as described herein.

As introduced above, the composition comprises a flavonoid component. Flavonoids (also called bioflavonoids) are natural botanical pigments that provide protection from free-radical damage, among other functions. Flavonoids occur in most fruits and vegetables. Examples of suitable flavonoids include, but are not limited to, quercetin, isoquercetin, hesperidin, rutin, naringin, naringenin, limonene, proanthocyanidins (e.g. A-type proanthocyanidins), and the like. The flavonoid component (e.g. Type A Proanthocyanidins) may be present in an amount of from 1 mg to 100 mg, alternatively from 10 mg to 90 mg, alternatively from 18 mg to 43 mg, alternatively from 18 mg to 36 mg, each based on a total weight of a serving of the composition. In various embodiments, the serving is adapted to be consumed by a human selected from the group of adults, juveniles, and a combination thereof. In certain embodiments, the serving is not adapted to be consumed by an infant.

The flavonoid component comprises an A-type proanthocyanidin (PAC). Without being bound by theory, it is believed that A-type linkages of such proanthocyanidins may promote binding of the A-type PAC to the FimH adhesin molecule at the tip of the fibrillum of E. coli and thereby competitively inhibit adherence to the binding site of urothelial glycoprotein receptors. In certain embodiments, the A-type proanthocyanidin may be derived from an extract of cranberry. In some embodiments, the flavonoid component comprising the A-type PAC is a cranberry extract, or derived from a cranberry extract. The A-type PAC may be present in an amount of at least 50 wt. %, alternatively at least 75 wt. %, alternatively at least 90 wt. %, each based on a total weight of the flavonoid component. In these or other embodiments, the composition may comprise any amount of the A-type PAC, such as from 0.1 to 40, alternatively from 0.25 to 10, alternatively from 0.5 to 5, wt. % of the A-type PAC, based on a total weight of the composition. It is to be appreciated that the particular amount of the A-type PAC present in the composition may vary in accordance with the total weight of the composition, which may be measured and/or described on the bases of a sample, a serving size, a batch, or an average of one or more samples, serving sizes, or batches. Typically, the amount of the A-type PAC present in the composition is measured and/or described with reference to a serving size of the composition as described herein.

Typically, the composition comprises quercetin. In some embodiments, the quercetin is provided as part of the flavonoid component described above. However, the quercetin may be provided in the composition in or as a separate component, such as a separate flavonoid component. For example, in some embodiments, the quercetin is provided in the composition as part of the flavonoid component comprising the A-type PAC. For example, in some embodiments, the quercetin and the A-type PAC may be provided in the form of, or otherwise derived from, one or more cranberry extracts. Typically, however, the quercetin is provided to the composition as a separate component, e.g. in the form of an isolate or purified quercetin.

Typically, the flavonoid component comprising quercetin is present in the composition in an amount of from 40 mg to 4,000 mg, alternatively from 45 mg to 3,000 mg, alternatively from 50 mg to 2,400 mg, based on a total weight of a serving of the composition. In some embodiments, the quercetin is present in an amount of from 50 mg to 200 mg, alternatively from 50 mg to 150 mg, alternatively from 75 mg to 125 mg.

It is to be appreciated that the particular amount of quercetin present in the composition may vary in accordance with the total weight of the composition, which may be measured and/or described on the bases of a sample, a serving size, a batch, or an average of one or more samples, serving sizes, or batches. Typically, the amount of quercetin present in the composition is measured and/or described with reference to a serving size of the composition as described herein.

As introduced above, the composition comprises a vitamin component, which comprises, alternatively consists essentially of, alternatively consists of ascorbic acid (i.e., vitamin C). Examples of suitable vitamins for inclusion in the vitamin component include, but are not limited to, vitamin A, vitamin B1, vitamin B2, vitamin B3, vitamin B5, vitamin B6, folic acid (vitamin B9), vitamin B12, vitamin B complex, vitamin D, vitamin D2, vitamin D3, vitamin E, vitamin G, vitamin H, vitamin K, vitamin M, vitamin 0, and vitamin Q10. The vitamin component may be present in an amount of from 10 mg to 300 mg, alternatively from 90 mg to 270 mg, alternatively from 144 mg to 216 mg, each based on a total weight of a serving of the composition. In various embodiments, the serving is adapted to be consumed by a human selected from the group of adults, juveniles, and a combination thereof. In certain embodiments, the serving is not adapted to be consumed by an infant.

The vitamin comprises the ascorbic acid. Without being bound by theory, it is believed that the ascorbic acid may distribute readily in high concentrations into immune cells, may have antimicrobial and natural killer cell activities, may promote lymphocyte proliferation, and may be consumed quickly during infections, thereby promoting an immune response to urinary tract infections. The ascorbic acid may be present in an amount of at least 50 wt. %, alternatively at least 75 wt. %, alternatively at least 90 wt. %, each based on a total weight of the vitamin component. In these or other embodiments, the composition may comprise any amount of the ascorbic acid, such as from 0.1 to 70, alternatively from 1 to 30, alternatively from 2 to 15, wt. % of the ascorbic acid, based on a total weight of the composition. It is to be appreciated that the particular amount of ascorbic acid present in the composition may vary in accordance with the total weight of the composition, which may be measured and/or described on the bases of a sample, a serving size, a batch, or an average of one or more samples, serving sizes, or batches. Typically, the amount of ascorbic acid present in the composition is measured and/or described with reference to a serving size of the composition as described herein.

As introduced above, the composition comprises a green tea extract. Examples of green tea extracts include extracts prepared from tea leaves of the Genus Camellia, for example, C. sinensis, C. assamica and the Yabukita variety, as well as hybrids thereof. Examples of prepared tea leaves include green teas such as sencha (i.e., middle-grade green tea), bancha (i.e., coarse green tea), gyokuro (i.e., shaded green tea), tencha (i.e., powdered tea) and kamairicha (i.e., roasted tea), and the like, as well as variations and combinations thereof. It is to be appreciated that the green tea extract used herein may be a concentrate, isolate, or other purified and/or processed form of an extraction product, and need not itself be the direct product of an extraction from green tea leaves. The green tea extract may be a full-spectrum extract, a selective extract, or a standardized extract. In some embodiments, the green tea extract comprises an extraction strength/extract ratio of from 4:1 to 12:1, such as from 5:1 to 11:1, alternatively from 6:1 to 10:1. In specific embodiments, the green tea extract is a 6-10/1 extract (i.e., comprises an extraction strength of from 6-10:1, as will be understood by those of skill in the art).

The green tea extract may be present in the composition in any amount such as from 5 mg to 100 mg, alternatively from 5 mg to 80 mg, alternatively from 10 mg to 80 mg, alternatively from 10 mg to 60 mg, alternatively from 15 mg to 60 mg, alternatively from 20 mg to 50 mg, alternatively from 25 mg to 40 mg, alternatively from 25 mg to 35 mg, based on a total weight of a serving of the composition. It is to be appreciated that the particular amount of the green tea extract present in the composition may vary in accordance with the total weight of the composition, which may be measured and/or described on the bases of a sample, a serving size, a batch, or an average of one or more samples, serving sizes, or batches. Typically, the amount of green tea extract present in the composition is measured and/or described with reference to a serving size of the composition as described herein. The extraction strength of the green tea extract may also be used in selecting an appropriate formulation amount. For example, a higher loading of a lower-strength extract may be utilized to provide the composition with the same, or similar, property from the green tea extract.

As introduced above, the composition optionally comprises a triterpenoid component, which comprises, alternatively consists essentially of, alternatively consists of a triterpenoid. Examples of suitable triterpenoids include, but are not limited to, pomolic acid, lantanolic acid, lantoic acid, camarin, lantacin, camarinin, ursolic acid, oleanolic acid, camaric acid, camarinic acid, azadirachtin H, lantanilic acid, bayogenin, hederagenin, medicagenic acid, zanhic acid, soyasapogenol B, beta-boswellic acid, and the like. The triterpenoid may be present in an amount of from 1.5 mg to 30 mg, alternatively from 7.5 mg to 22.5 mg, alternatively from 12 mg to 18 mg, each based on a total weight of a serving of the composition. In various embodiments, the serving is adapted to be consumed by a human selected from the group of adults, juveniles, and a combination thereof. In certain embodiments, the serving is not adapted to be consumed by an infant.

In some embodiments, the triterpenoid component comprises, alternatively consists essentially of, alternatively consists of ursolic acid. Ursolic acid is a pentacyclic triterpenoid derived from sage, cranberries, apples, waxy berries, rosemary, oregano, and several other plants and herbs. Without being bound by theory, it is believed that the ursolic acid may reduce inflammatory symptoms resulting from urinary tract infections by E. coli. Further, the ursolic acid may inhibit E. coli biofilm formation. In certain embodiments, the ursolic acid may be derived from an extract of sage leaf. The ursolic acid may be present in an amount of at least 50 wt. %, alternatively at least 75 wt. %, alternatively at least 90 wt. %, each based on a total weight of the triterpenoid component. In these or other embodiments, the composition may comprise any amount of the ursolic acid, such as from 0.01 to 20, alternatively from 0.1 to 10, alternatively from 0.25 to 5, wt. % of the ursolic acid, based on a total weight of the composition. It is to be appreciated that the particular amount of ursolic acid present in the composition may vary in accordance with the total weight of the composition, which may be measured and/or described on the bases of a sample, a serving size, a batch, or an average of one or more samples, serving sizes, or batches. Typically, the amount of ursolic acid present in the composition is measured and/or described with reference to a serving size of the composition as described herein.

In some embodiments, the ursolic acid is provided to the composition in combination with one or more of the components described herein, such that the triterpenoid component need not be a stand-alone or isolable component of the composition. For example, in some embodiments, the ursolic acid is provided in the composition as part of a combination comprising the flavonoid component described above. In particular embodiments, the ursolic acid, the quercetin, and the A-type PAC are provided in the form of, or otherwise derived from, a cranberry extract. In yet other embodiments, the composition is free from, alternatively substantially free from, ursolic acid. In yet other embodiments, the composition comprises quercetin and the A-type PAC from a cranberry extract, and the ursolic acid from a sage leaf extract.

In various embodiments, the composition comprises a sweetener, which is used to improve taste or palatability. While those which are commercially available are preferred, this description does not preclude those sweeteners which are not in common use, commercially available or the like. Examples of suitable sweeteners include, but are not limited to sucrose, fructose, extracts of certain plants such as mung-fruit, stevia, or the like, sugar alcohols such as sucralose and the like, and combinations thereof.

In some instances, the sweetener may also act as a functional component of the composition. For example, in some embodiments, the composition comprises a fructose. Without being bound by theory, it is believed that the fructose improves binding affinity of the D-mannose to the adhesin FimH s molecule at the tip of the fibrillum of E. coli. As a result of this theory, the sweetener, such as the fructose, may be utilized in the same fashion, though fructose is not often favored by consumers as a sweetener in certain areas. The sweetener may be present in any amount that provides acceptable taste to a subject consuming the composition (e.g. when formulated for consumption). Generally, the amount of sweetener ranges from 100 mg to 5,000 mg, alternatively from 500 mg to 3,000 mg, alternatively from 1,000 mg to 2,000 mg, each based on a total weight of a serving of the composition.

In various embodiments, the serving is adapted to be consumed by a human selected from the group of adults, juveniles, and a combination thereof. In certain embodiments, the serving is not adapted to be consumed by an infant. In certain embodiments, the composition may comprise any amount of sweetener, such as from 0.01 to 50, alternatively from 0.1 to 20, alternatively from 0.1 to 10, wt. % of the sweetener, based on the total weight of the composition. It is to be appreciated that the particular amount of sweetener, or any component in the composition that is present in the composition may vary in accordance with the total weight of the composition, which may be measured and/or described on the bases of a sample, a serving size, a batch, or an average of one or more samples, serving sizes, or batches. Typically, the amount of sweetener present in the composition is measured and/or described with reference to a serving size of the composition as described herein.

The composition may comprise a synergistic combination of the HMO and the D-mannose. The synergy of the HMO and the D-mannose in the context of the present embodiments may be assessed qualitatively and/or quantitatively. For example, quantitatively assessment of the synergy may include assaying anti-microbial activity of the components and composition towards uro- and enteropathogenic E. coli, e.g. via ELISA with isolated bacterial adhesins (e.g. FimH adhesins as described above).

The HMO and the D-mannose may be present in the composition in a weight ratio of the HMO to the D-mannose from 1:100 to 100:1, alternatively from 1:10 to 10:1, alternatively from 1:5 to 5:1, alternatively from 1:4 to 4:1, alternatively from 1:3 to 3:1, alternatively from 1:2 to 2:1 (HMO:D-mannose).

In various embodiments, the D-mannose and the HMO are present in the composition in a weight ratio of the HMO to the D-mannose of from 1:100 to 90:1, such as from 1:100 to 80:1, alternatively from 1:100 to 70:1, alternatively from 1:100 to 60:1, alternatively from 1:100 to 50:1, alternatively from 1:100 to 40:1, alternatively from 1:100 to 30:1, alternatively from 1:100 to 20:1, alternatively from 1:100 to 10:1, alternatively from 1:100 to 1:1 (HMO:D-mannose).

In various embodiments, the D-mannose and the HMO are present in the composition in a weight ratio of the HMO to the D-mannose of from 1:90 to 100:1, such as from 1:80 to 100:1, alternatively from 1:70 to 100:1, alternatively from 1:60 to 100:1, alternatively from 1:50 to 100:1, alternatively from 1:40 to 100:1, alternatively from 1:30 to 100:1, alternatively from 1:20 to 100:1, alternatively from 1:10 to 100:1, or alternatively from 1:1 to 100:1 (HMO:D-mannose).

In exemplary embodiments, the D-mannose and the HMO are present in the composition in a weight ratio of the HMO to the D-mannose from 1:50 to 6:1, such as from 1:50 to 1:30 (e.g. 1:42 and 1:40), alternatively from 1:40 to 1:20 (e.g. 1:30), alternatively from 1:30 to 1:10 (e.g. 1:21), alternatively from 1:9 to 1:7 (e.g. 10:82 and 1:8), alternatively from 1:6 to 1:4 (e.g. 1:5), alternatively from 1:3 to 1:1 (e.g. 1:2), alternatively from 4:1 to 6:1 (e.g. 5:1) (HMO:D-mannose).

The composition may comprise one or more additional components, such as those described as optional above, as well as those described below. Examples of suitable additional components include, but are not limited to, additive components (e.g. flavoring agents, colorants, fillers, binders, preservatives, etc.), amino acids, peptides, proteins, lipids, vitamins, carbohydrates, nucleic acids, minerals, anabolic nutrients, antioxidants, probiotic bacterial strains, lipotropic agents, and combinations thereof. The composition may include the additional components, collectively or individually, in an amount of from 0.01 to 75, alternatively from 1 to 75, alternatively from 1 to 50, wt. %, each based on a total weight of the composition.

In some embodiments, the composition comprises one or more additive components.

In such embodiments, the additive component typically comprises a flavoring agent, a dye, a flow modifier, a preservative, a filler, a binder, a dispersing agent, a carrier, a supplemental nutrient, or a combination thereof. The composition may include the additive component(s), individually or collectively, in an amount of from 0.01 to 75, alternatively from 1 to 75, alternatively from 1 to 50, wt. %, each based on a total weight of the composition.

In general, the composition itself may be formulated and/or prepared in any amount, e.g. suitable for but a single dose/serving or instead in a bulk or batch form comprising multiple servings. For example, as will be appreciated from the description herein, the composition may comprise a serving size of from 250 mg to 10,000 mg, such as from 500 mg to 7,500 mg, alternatively from 1000 mg to 7,000 mg, alternatively from 2,500 mg to 5,000 mg, alternatively of from 2,500 mg to 4,500 mg, alternatively of from 2,500 mg to 4,000 mg, alternatively of from 2,000 mg to 4,000 mg. It will be appreciated that, depending on the particular formulation, the serving size of the composition may be outside of these ranges as well. For example, when relatively large amounts of sweetener and/or other additives are utilized, a serving of the composition may be on the scale of from 10 to 25 g, or even more. Moreover, the composition may comprise a total weight higher than those listed above, e.g. when prepared in bulk form for use in multiple servings.

The composition may compose any form, such as a dry powder, a solution, a suspension, an emulsion, or the like. In certain embodiments, the composition is a dry powder.

In some embodiments, the composition is adapted to be consumed as a liquid. For example, the composition may be a dry powder that is combined with a consumable liquid (e.g. water) to form a consumable liquid solution, suspension, or emulsion comprising the composition. Likewise, the composition may be adapted to be mixed with a foodstuff or beverage. As such, in some embodiments, the composition is, alternatively is a component of, a foodstuff or beverage. In these or other embodiments, the composition may be further defined as a food additive. Accordingly, it is to be appreciated that certain aspects of the present embodiments include the use of the composition as a food additive, and the use of the composition in methods of preparing foodstuff and/or beverages.

As introduced above, the present embodiments encompass and include a foodstuff or beverage comprising the composition. The foodstuff or beverage is typically suitable for use in methods of treating, ameliorating, or preventing an infection in a mammalian urinary tract or a cause or symptoms thereof.

Typically, the foodstuff or beverage comprises an admixture of the composition with one or more feed products, liquids, supplements, or combinations thereof. For example, in particular embodiments, the amount of the composition in the foodstuff or beverage comprises from 1 gram to 12 grams, alternatively from 2 grams to 8 grams, alternatively from 2 grams to 4.5 grams, of the composition per serving/unit dose of the foodstuff or beverage, depending on the subject, the malady, the severity of the condition which is often determined under the supervision a skilled medical practitioner. However, in certain embodiments the composition may itself be further defined as a foodstuff or beverage composition, depending on the quantity, nature, and identity of individual additives and components present in the composition, such as those described above. Said differently, it will be appreciated that the feed products above may be the same as, or different from, the additional and/or additive components described above. As such, the formulation of the composition in terms of peripheral components may be selected to impart a particular form to the composition as a whole, such that the composition itself may be formulated as the foodstuff or beverage (or other particular forms described herein). Thus, it is to be appreciated that the embodiments described herein with respect to the composition are intended to equally encompass the foodstuff or beverage, a food or beverage product, and/or a food supplement comprising the composition. Accordingly, any amounts and/or examples of such components described herein with respect to the composition itself may equally apply to the foodstuff or beverage comprising the composition, as will be understood by one of skill in the art.

In some embodiments, the foodstuff or beverage comprising the composition is further defined as a nutritional composition. The nutritional composition typically has a nutritional value of at least 1 kilocalorie (kcal) per 100 grams (g) for dry food formulations (i.e., foodstuffs), or per 100 milliliters (ml) for liquid formulations (i.e., beverages). In certain embodiments, the nutritional composition has a nutritional value of at least 10, alternatively at least 50, alternatively at least 100, alternatively at least 300, kcal per 100 g for dry food formulations (i.e., foodstuffs), or per 100 ml for liquid formulations (i.e., beverages). In at least one embodiment, the nutritional formulation has a nutritional value of from 50 to 200 kcal/100 ml for liquid formulations, and of from 300 to 600 kcal/100 g for dry food formulations. In these or other embodiments, the nutritional composition is in the form of a dry food concentrate, which may be mixed with liquid or food and subsequently consumed. It is to be appreciated that the nutritional composition is distinguished from a vaccine, and the compositions described herein may be free, alternatively substantially free, from a vaccine.

In addition to the components described above with respect to the composition and the foodstuff or beverage comprising the composition, the nutritional composition may further comprise ingredients selected from lipids, minerals, carbohydrates, amino acids, amino acid chelates, anabolic nutrients, vitamins, antioxidants, probiotic bacterial strain, lipotropic agents, and the like, which may each be independently selected in order to provide the nutritional composition with a formulation capable of sustaining energy and/or anabolism in an animal.

In certain embodiments, the nutritional composition may be further defined as a nutritional supplement, or as a complete nutritive. For example, the nutritional composition may be formulated to provide a mammal (e.g. a human), via consumption of the nutritional composition, with at least 5%, alternatively at least 10%, alternatively at least 25%, alternatively at least 50%, alternatively at least 75%, alternatively at least 90%, of daily calories required by the mammal. However, it is to be appreciated that a daily calorie requirement is dependent on several factors, including the gender, height, and/or age of the mammal, and thus the percentage of caloric requirement provided by the nutritional composition will be dependent on the particular person consuming the nutritional composition. For example, a 30 year old human male of 80 kg body weight and 180 cm height has a daily calorie requirement of around 2900 cal (calories) to maintain his body weight whereas a 30 year old human female of 55 kg body weight and 165 cm height has a daily calorie requirement of around 2100 cal to maintain her body weight. In at least one embodiment, the nutritional formulation is a nutritional product for human juveniles or adults.

In certain embodiments, the foodstuff or beverage is further defined as an animal food. In such embodiments, the foodstuff or beverage is typically formulated for ingestion by one or more non-human animals, such as livestock including cattle, swine, horses, sheep, goats, poultry, and fish, domesticated companionship species such as dogs, cats, fish, and rodents, undomesticated wildlife such as deer, moose, elk, migratory, and non-migratory fowl, those non-human animals described herein, and combinations thereof.

In some embodiments, the foodstuff or beverage is further defined as a medical food. As such, it is to be appreciated that the medical food comprises the composition, and may be the same as or different from the nutritional composition described above.

As introduced above, a method of treating, ameliorating, or preventing an infection in a urinary tract of a mammal is provided. The method comprises administering the composition to the mammal. In particular, the composition described herein has surprisingly been found to be useful as a therapeutic for treating, ameliorating, or preventing an infection in a urinary tract of a mammal. As such, the composition may be used in this fashion (e.g. in a step of the method) by administering the composition to a mammal in need thereof. However, it is to be appreciated that the composition may be useful in a method of, or as a therapeutic for, treating, ameliorating, or preventing any infection of a mammal.

As also introduced above, a combination for use in a method of treating, ameliorating, or preventing an infection in a mammalian urinary tract is further provided. The combination comprises a D-mannose and an isolated human milk oligosaccharide (HMO), which are typically provided in the combination as described above concerning the composition. In certain embodiments, the combination further comprises an A-type proanthocyanidin and quercetin. In some such embodiments, the combination further comprises ascorbic acid. In particular such embodiments, the combination further comprises ursolic acid.

Typically, the D-mannose is present in a serving of the combination in an amount of from 100 mg to 4,000 mg, such as from 1,000 mg to 3,000 mg, alternatively from 1,600 mg to 2,400 mg, and the HMO is present in the serving of combination in an amount of from 100 mg to 1,000 mg, such as from 100 mg to 400 mg, alternatively from 125 mg to 400 mg, alternatively from 125 mg to 300 mg, alternatively from 150 mg to 300 mg. In such embodiments, the A-type proanthocyanidin (when utilized) is typically present in the serving of the combination in an amount of from 10 mg to 100 mg, such as from 18 mg to 90 mg, alternatively from 18 mg to 43 mg. The quercetin (when utilized) is typically present in the serving of the combination in an amount of from 50 mg to 150 mg, such as from 75 mg to 125 mg, alternatively from 90 mg to 110 mg. The ascorbic acid (when utilized) is typically present in the serving of the combination in an amount of from 10 mg to 300 mg, such as from 90 mg to 270 mg, alternatively from 144 mg to 216 mg. The green tea extract (when utilized) is typically present in the serving of the combination in an amount of from 5 mg to 100 mg, such as from 5 mg to 80 mg, alternatively from 10 mg to 80 mg, alternatively from 10 mg to 60 mg, alternatively from 15 mg to 60 mg, alternatively from 20 mg to 50 mg, alternatively from 25 mg to 40 mg, alternatively from 25 mg to 35 mg. The ursolic acid (when utilized) is typically present in the serving of the combination in an amount of from 1.5 mg to 30 mg, such as from 7.5 mg to 22.5 mg, alternatively from 12 mg to 18 mg. As described above with respect to the composition, the serving of the combination may be adapted to be consumed by a human selected from the group of adults, juveniles, and a combination thereof. Likewise, the serving of the combination may not be adapted to be consumed by an infant.

In various embodiments, treating, ameliorating, or preventing an infection in a mammalian urinary tract (e.g. with the composition or combination, or via the method) comprises improving or enhancing the quality of life of the mammal. In some such embodiments, the mammal is a human and the method of treating, ameliorating, or preventing an infection in a mammalian urinary tract comprises improving or enhancing the quality of life of the human.

It is to be appreciated that the composition may be administered to the animal by any means known in the art, including via topical, enteral, or parenteral routes. Typically, the composition is administered orally. However, rectal and/or enteral administration may also be used.

The composition may be administered as needed, daily, several times per day or in any suitable regimen such that the desired outcome is achieved. In the methods, the frequency of administration can depend on several factors, including the desired level of prevention and/or treatment. Generally, a regimen includes administration of the composition to the host once or twice daily to include an administration in the morning and/or an administration in the evening. The amount of composition administered to the host during each administration may depend on several factors including level of desired results and the specific composition.

The composition may be assessed for efficacy or otherwise, e.g. via evaluating for general taste and tolerance by patients, etc. Likewise, the composition may be evaluated via trial/test in a small human population, via comparison to a placebo, etc. For example, the composition may be tested in a study involving a population of healthy human females having had one or more UTIs in the previous 12 months, more than two UTIs in the previous three years, etc. Exclusion criteria may include, for example, current UTI or treatment for UTI at the time of the study. Additional selection criteria may also be utilized, such as cystitis, recurring UTIs (e.g. resolvable via single course of antibiotics), fatigue, diffuse pain, iron deficiency, as well as general or localized dysbiosis (e.g. imbalance or deficiency in a subject's bladder microbiota, gut microbiota, vaginal microbiota, urethral microbiota, etc.).

Once identified, a patient may be evaluated for symptoms before beginning treatment (T=0) and at recurring intervals thereafter (e.g. 4 weeks, 12 weeks, 16 weeks, etc.). Such evaluation may include urinalysis and/or microbiome analysis, e.g. for the presence of E. coli or implicated in UTIs, which may include quantifying colony forming units per g (cfu/g), UTI occurrence (and concomitant discontinuation of the study), changes in quality of life, and urinary symptoms.

Treatment may include administration of the composition (e.g. via ingestion of one of the forms of the nutritional composition described above) on a consistent dosing regimen (e.g. one or two servings per day) until alleviation, reduction, or cessation of symptoms is evident. Further administration of the composition may also be used, at the same or lower frequency from the initial treatment for maintenance and/or prevention. For example, a serving of the composition may be administered twice daily during an initial 12-week treatment period, followed thereafter by administering a serving once daily for maintenance of a reduced or symptom-free state.

Having generally described the compositions of this disclosure, the following are examples of those compositions for further illustration.

### Examples 1-13: Dry Composition Dosage Forms

Various compositions are prepared in accordance with the embodiments above to give Examples 1-13, the particular components and parameters of which are shown in Tables 1 & 2 below. Specifically, for the composition examples below (abbreviated in the Tables as “Ex.”), the following commercially available components are used: 1) a sage leaf extract having 15% ursolic acid (Ursolic Acid); 2) commercially available human milk oligosaccharides (HMO) 2′-O-fucosyllactose (2′-FL) and/or 3′-O-sialyllactose (3′-SL) (though others can be used, e.g. at lab scale); 3) a 6-10/1 green tea extract; and 4) commercially available sweeteners cane sugar (“SUC”), fructose (“FRUC”), stevia (“STEV”) and sucralose (“SLOS”). The table below illustrates formulation of a dry form dosage that could be added to a liquid beverage (water, juice, etc.). All amounts are recited in milligrams (mg), including components that have gram quantities involved:

TABLE 1
Components and Parameters of Examples 1-7

| Ingredient | | Ex. 1 | Ex. 2 | Ex. 3 | Ex. 4 | Ex. 5 | Ex. 6 | Ex. 7 |
|---|---|---|---|---|---|---|---|---|
| PAC-A (mg): | | 36 | 50 | 72 | 72 | 40 | 50 | 18 |
| HMO | 2′-FL | 50 | — | 100 | — | 50 | — | 500 |
| | (mg): | | | | | | | |
| | 3′-SL | — | 60 | — | 100 | 50 | 50 | — |
| | (mg): | | | | | | | |
| D-mannose (mg): | | 2000 | 2500 | 500 | 2100 | 500 | 2000 | 100 |
| Ursolic | | 25 | 150 | 50 | 50 | 150 | 100 | 10 |
| Acid (mg): | | | | | | | | |
| Ascorbic | | 250 | 100 | 200 | 100 | 150 | 150 | 300 |
| Acid (mg): | | | | | | | | |
| Sweetener | FRUC | 20 | — | — | 15 | — | — | 25 |
| | (mg): | | | | | | | |
| | STEV | — | 25 | — | 10 | 20 | 25 | — |
| | (mg): | | | | | | | |
| | SLOS | — | — | 30 | — | — | — | — |
| | (mg): | | | | | | | |
| | SUC | — | — | — | — | — | — | — |
| | (mg): | | | | | | | |

TABLE 2
Components and Parameters of Examples 8-13

| Ingredient | | Ex. 8 | Ex. 9 | Ex. 10 | Ex. 11 | Ex. 12 | Ex. 13 |
|---|---|---|---|---|---|---|---|
| PAC-A (mg): | | 100 | 50 | 274 | 36 | 18 | 18 |
| HMO | 2′-FL | — | 1000 | 255 | 250 | 150 | 150 |
| | (mg): | | | | | | |
| | 3′-SL | 100 | — | — | — | — | — |
| | (mg): | | | | | | |
| D-mannose (mg): | | 3000 | 2000 | 2100 | 2000 | 2000 | 2000 |
| Ursolic | | 200 | 10 | 105 | 100 | — | — |
| Acid (mg): | | | | | | | |
| Ascorbic | | 270 | 10 | 100 | 180 | 180 | 180 |
| Acid (mg): | | | | | | | |
| Green Tea | | 30 | | | | 30 | 30 |
| Extract (mg): | | | | | | | |
| Sweetener | FRUC | 15 | — | — | — | — | — |
| | (mg): | | | | | | |
| | STEV | — | — | 10 | 10 | 7.5 | 7.5 |
| | (mg): | | | | | | |
| | SLOS | — | 10 | 20 | 20 | — | — |
| | (mg): | | | | | | |
| | SUC | 10 | — | — | — | — | 17.5 |
| | (mg): | | | | | | |

The compositions are prepared by combining and dry blending the ingredients together into a homogenous mixture. Each composition is then packaged in bulk according to the example formulation listed. Example formulations may be packaged into individual dosage containers, sachets, etc.

### Example 14: Dry Composition

A composition is prepared in accordance with the embodiments above to give Example 14, the particular components and parameters of which are shown in Table 3 below. Specifically, the ingredients are combined together to form a mixture, which is dry blended to give the composition as a homogenous mixture.

TABLE 3
Components and Parameters of Example 14

| | | % | % | Active |
|---|---|---|---|---|
| | Amount, | Active | Overage | Amount |
| Ingredient | wt. (mg) | (%) | (%) | (mg) |
| D-mannose: | 2081.633 | 98.00 | 2.00 | 2000.00 |
| HMO (2′-FL): | 153.000 | 100.00 | 2.00 | 150.00 |
| Cranberry PE | 136.957 | 13.80 | 5.00 | 18.00 |
| (15% PAC | | | | |
| BL-DMAC): | | | | |
| Quercetin | 110.526 | 95.00 | 5.00 | 100.00 |
| Ascorbic Acid: | 209.091 | 99.00 | 15.00 | 180.000 |
| Green Tea | 31.500 | 100.00 | 5.00 | 30.00 |
| Extract 6-10/1: | | | | |
| Stevia RS 97/97: | 7.500 | 100.00 | 0.00 | 7.50 |
| Sucralose | 17.500 | 100.00 | 0.00 | 17.50 |
| Powder-FP: | | | | |
| Citric Acid, | 125.000 | 100.00 | 0.00 | 125.00 |
| Anhydrous: | | | | |
| Cranberry Flavor: | 200.000 | 100.00 | 0.00 | 200.000 |
| Total: | 3072.71 | — | — | 2828.00 |

The composition of Example 14 is provided with amounts shown on a per-dose basis. As such, the example formulation may be packaged into individual dosage containers, sachets, etc., or bulk-packaged into a multi-dose container.

The following additional embodiments are provided, the numbering of which is not to be construed as designating levels of importance.

Embodiment 1 relates to a composition comprising: a monosaccharide comprising a D-mannose; an oligosaccharide comprising an isolated human milk oligosaccharide (HMO), a flavonoid comprising an A-type proanthocyanidin, a triterpenoid comprising ursolic acid, and a vitamin comprising ascorbic acid.

Embodiment 2 relates to the composition Embodiment 1, wherein the composition comprises fructose, and wherein the fructose is present in an amount of from 100 mg to 5,000 mg, optionally from 500 mg to 3,000 mg, optionally from 1,000 mg to 2,000 mg, each based on a total weight of a serving of the composition, wherein the serving is adapted to be consumed by a human selected from the group of adults, juveniles, and a combination thereof, and wherein the serving is not adapted to be consumed by an infant.

Embodiment 3 relates to the composition Embodiment 1 or 2, wherein the composition comprises an amino acid, a peptide, a protein, a lipid, a vitamin, a carbohydrate, a nucleic acid, a mineral, an anabolic nutrient, an antioxidant, a probiotic bacterial strain, a lipotropic agent, or combinations thereof.

Embodiment 4 relates to a method of treating, ameliorating, or preventing an infection in a urinary tract of a mammal, the method comprising administering a composition according to any one of the previous Embodiments to the mammal.

Embodiment 5 relates to a foodstuff or beverage comprising a composition according to any one of the previous Embodiments.

Embodiment 6 relates to the foodstuff or beverage of Embodiment 5, wherein the foodstuff or beverage is further defined as a medical food.

Embodiment 7 relates to a method of treating or preventing urinary tract infection comprising administering to a mammal an effective amount of a composition comprising a human milk oligosaccharide.

Embodiment 8 relates to a composition useful for the method of Embodiment 7.

Embodiment 9 relates to a use of human milk oligosaccharides in the manufacture of a formulation for the treatment of (or prevention of) urinary tract infection.

Embodiment 10 relates to a method of treating or preventing urinary tract infection comprising administering to a mammal an effective amount of a composition comprising a human milk oligosaccharide, wherein the human milk oligosaccharide is selected from the group comprising 2′-O-fucosyllactose (2′-FL), 3-fucosyllactose (3-FL), 3′-O-sialyllactose (3′-SL), 6′-O-sialyllactose (6′-SL), lacto-N-tetraose (LNT), lacto-N-neotetraose (LNnT), lacto-N-hexaose (LNH), iso-lacto-N-octaose, iso-lacto-N-neooctaose, para-lacto-N-octaose, lacto-N-fucopentaose I (LNFP I), lacto-N-fucopentaose II (LNFP II), lacto-N-fucopentaose III (LNFP III), lacto-N-fucopentaose V (LNFP V), LS-tetrasaccharide a (LST a), LS-tetrasaccharide b (LST b), LS-tetrasaccharide c (LST c), disialyliacto-N-tetraose (DSLNT), difucosyllactose (DFL), and combinations thereof.

Embodiment 11 relates to a method of treating or preventing urinary tract infection comprising administering to a mammal an effective amount of a composition comprising a human milk oligosaccharide, wherein the human milk oligosaccharide is selected from the group comprising 2-O-fucosyllactose (2′-FL), 3-fucosyllactose (3-FL) and combinations thereof.

Embodiment 12 relates to a method of treating or preventing urinary tract infection comprising administering to a mammal an effective amount of a composition comprising a human milk oligosaccharide and D-mannose.

Embodiment 13 relates to a method of treating or preventing urinary tract infection comprising administering to a mammal an effective amount of a composition comprising a human milk oligosaccharide, wherein the human milk oligosaccharide is selected from the group comprising 2-O-fucosyllactose (2′-FL), 3-fucosyllactose (3-FL) and combinations thereof, and D-mannose.

Embodiment 14 relates to a method of treating or preventing urinary tract infection comprising administering to a mammal an effective amount of a composition comprising a human milk oligosaccharide and one or more flavonoids comprising an A-type proanthocyanidin (PAC).

Embodiment 15 relates to a method of treating or preventing urinary tract infection comprising administering to a mammal an effective amount of a composition comprising a human milk oligosaccharide and an effective amount of ascorbic acid.

Embodiments 16 relates to a combination for use in a method of treating, ameliorating, or preventing an infection in a mammalian urinary tract, the combination comprising D-mannose and an isolated human milk oligosaccharide (HMO).

Embodiment 17 relates to the combination of claim 16, wherein the combination comprises A-type proanthocyanidin, ursolic acid, and ascorbic acid.

Embodiment 18 relates to a composition comprising: a monosaccharide comprising a D-mannose; an oligosaccharide comprising an isolated human milk oligosaccharide (HMO); a flavonoid comprising an A-type proanthocyanidin; quercetin; a vitamin comprising ascorbic acid; and a green tea extract.

Embodiment 19 relates to the composition of Embodiment 18, wherein: (i) the D-mannose is present the monosaccharide in an amount of at least 50 wt. %, optionally at least 75 wt. %, optionally at least 90 wt. %, based on a total weight of the monosaccharide; (ii) the monosaccharide comprising the D-mannose is present in the composition in an amount of from 100 mg to 4,000 mg, optionally from 1,000 mg to 3,000 mg, optionally from 1,600 mg to 2,400 mg based on a serving of the composition adapted to be consumed by a human selected from the group of adults, juveniles, and a combination thereof, wherein the serving is not adapted to be consumed by an infant; or (iii) both (i) and (ii).

Embodiment 20 relates to the composition of Embodiment 18 or 19, wherein: (i) the isolated HMO is present in the oligosaccharide in an amount of at least 50 wt. %, optionally at least 75 wt. %, optionally at least 90 wt. %, based on a total weight of the oligosaccharide; (ii) the oligosaccharide comprising the isolated HMO is present in the composition in an amount of from 100 mg to 1,000 mg, optionally from 100 mg to 400 mg, optionally from 125 mg to 400 mg, optionally from 125 mg to 300 mg, optionally from 150 mg to 300 mg based on a serving of the composition adapted to be consumed by a human selected from the group of adults, juveniles, and a combination thereof, wherein the serving is not adapted to be consumed by an infant; (iii) the isolated HMO is selected from the group of 2′-O-fucosyllactose (2′-FL), 3-fucosyllactose (3-FL), 3-O-sialyllactose (3′-SL), 6-O-sialyllactose (6′-SL), lacto-N-tetraose (LNT), lacto-N-neotetraose (LNnT), lacto-N-hexaose (LNH), iso-lacto-N-octaose, iso-lacto-N-neooctaose, para-lacto-N-octaose, lacto-N-fucopentaose I (LNFP I), lacto-N-fucopentaose II (LNFP II), lacto-N-fucopentaose III (LNFP III), lacto-N-fucopentaose V (LNFP V), LS-tetrasaccharide a (LST a), LS-tetrasaccharide b (LST b), LS-tetrasaccharide c (LST c), disialyliacto-N-tetraose (DSLNT), difucosyllactose (DFL), and combinations thereof; or (iv) any combination of (i)-(iii).

Embodiment 21 relates to the composition of any one of Embodiments 18-20, wherein: (i) the A-type proanthocyanidin is present in the flavonoid comprising the A-type proanthocyanidin in an amount of at least 50 wt. %, optionally at least 75 wt. %, optionally at least 90 wt. %, based on a total weight of the flavonoid comprising the A-type proanthocyanidin; (ii) the flavonoid component comprising the A-type proanthocyanidins is present in an amount of from 1 mg to 100 mg, alternatively from 10 mg to 90 mg, alternatively from 18 mg to 43 mg, alternatively from 18 mg to 36 mg based on a serving of the composition adapted to be consumed by a human selected from the group of adults, juveniles, and a combination thereof, wherein the serving is not adapted to be consumed by an infant; (iii) the flavonoid comprising the A-type proanthocyanidin is derived from an extract of cranberry; or (iv) any combination of (i)-(iii).

Embodiment 22 relates to the composition of any one of Embodiments 18-21, wherein quercetin is present in the composition in an amount of from 40 mg to 4,000 mg, optionally from 45 mg to 3,000 mg, optionally from 50 mg to 2,400 mg, optionally from 50 mg to 150 mg based on a serving of the composition adapted to be consumed by a human selected from the group of adults, juveniles, and a combination thereof, wherein the serving is not adapted to be consumed by an infant.

Embodiment 23 relates to the composition of any one of Embodiments 18-22, wherein: (i) the ascorbic acid is present in the vitamin in an amount of at least 50 wt. %, optionally at least 75 wt. %, optionally at least 90 wt. %, based on a total weight of the vitamin; (ii) the vitamin comprising ascorbic acid is present in the composition in an amount of from 10 mg to 300 mg, optionally from 90 mg to 270 mg, optionally from 144 mg to 216 mg based on a total weight of a serving of the composition adapted to be consumed by a human selected from the group of adults, juveniles, and a combination thereof, and wherein the serving is not adapted to be consumed by an infant; or (iii) both (i) and (ii).

Embodiment 24 relates to the composition of any one of Embodiments 18-23, wherein: (i) the green tea extract is a 6-10/1 extract; (ii) the green tea extract is present in the composition in an amount of from 15 mg to 60 mg, optionally from 20 mg to 50 mg, optionally from 25 mg to 40 mg, optionally from 25 mg to 35 mg based on a total weight of a serving of the composition adapted to be consumed by a human selected from the group of adults, juveniles, and a combination thereof, and wherein the serving is not adapted to be consumed by an infant; or (iii) both (i) and (ii).

Embodiment 25 relates to the composition of any one of Embodiments 18-24, further comprising a triterpenoid comprising ursolic acid.

Embodiment 26 relates to the composition of Embodiment 25, wherein: (i) the triterpenoid comprising ursolic acid is derived from an extract of sage leaf; (ii) the ursolic acid is present in the triterpenoid in an amount of at least 50 wt. %, optionally at least 75 wt. %, optionally at least 90 wt. %, based on a total weight of the triterpenoid; (iii) the triterpenoid comprising ursolic acid is present in an amount of from 1.5 mg to 30 mg, optionally from 7.5 mg to 22.5 mg, optionally from 12 mg to 18 mg, based on a serving of the composition adapted to be consumed by a human selected from the group of adults, juveniles, and a combination thereof, and wherein the serving is not adapted to be consumed by an infant; or (iv) any combination of (i)-(iii).

Embodiment 27 relates to the composition of any one of Embodiments 18-26, wherein a serving of the composition comprises a total weight of from 250 mg to 10,000 mg, optionally from 500 mg to 7,500 mg, optionally from 1000 mg to 7,000 mg, optionally from 2,500 mg to 5,000 mg and is adapted to be consumed by a human selected from the group of adults, juveniles, and a combination thereof, and wherein the serving is not adapted to be consumed by an infant.

Embodiment 28 relates to a combination for use in a method of treating, ameliorating, or preventing an infection in a mammalian urinary tract, the combination comprising the composition of any one of Embodiments 18-27.

Embodiment 29 relates to the combination of Embodiment 28, wherein the D-mannose is present in an amount of from 100 mg to 4,000 mg, optionally from 1,000 mg to 3,000 mg, optionally from 1,600 mg to 2,400 mg per serving of the combination.

Embodiment 30 relates to the combination of any one of Embodiments 28-29, wherein the HMO is present in an amount of from 100 mg to 1,000 mg, optionally from 100 mg to 400 mg, optionally from 150 mg to 300 mg per serving of the combination.

Embodiment 31 relates to the combination of any one of Embodiments 28-30, wherein the A-type proanthocyanidin is present in an amount of from 10 mg to 100 mg, optionally from 15 mg to 90 mg, optionally from 18 mg to 43 mg per serving of the combination.

Embodiment 32 relates to the combination of any one of Embodiments 28-31, wherein the quercetin is present in an amount of from 40 mg to 4,000 mg, optionally from 45 mg to 3,000 mg, optionally from 50 mg to 2,400 mg per serving of the combination.

Embodiment 33 relates to the combination of any one of Embodiments 28-32, wherein the ascorbic acid is present in an amount of from 10 mg to 300 mg, optionally from 90 mg to 270 mg, optionally from 144 mg to 216 mg per serving of the combination.

Embodiment 34 relates to the combination of any one of Embodiments 28-33, wherein the green tea extract is present in an amount of from 15 mg to 60 mg, optionally from 20 mg to 50 mg, optionally from 25 mg to 40 mg, optionally from 25 mg to 35 mg per serving of the combination.

Embodiment 35 relates to the combination of one of Embodiments 28-34, wherein the combination comprises the triterpenoid comprising ursolic acid, where the ursolic acid is present in an amount of from 1.5 mg to 30 mg, optionally from 7.5 mg to 22.5 mg, optionally from 12 mg to 18 mg per serving of the combination.

Embodiment 36 relates to the combination of any one of Embodiments 28-35, wherein, per serving of the combination: (i) the D-mannose is present in an amount of from 100 mg to 4,000 mg, optionally from 1,000 mg to 3,000 mg, optionally from 1,600 mg to 2,400 mg; (ii) the HMO is present in an amount of from 100 mg to 1,000 mg, optionally from 100 mg to 400 mg, optionally from 150 mg to 300 mg; (iii) the A-type proanthocyanidin is present in an amount of from 10 mg to 100 mg, optionally from 15 mg to 90 mg, optionally from 18 mg to 43 mg; (iv) the quercetin is present in an amount of from 40 mg to 4,000 mg, optionally from 45 mg to 3,000 mg, optionally from 50 mg to 2,400 mg; (v) the ascorbic acid is present in an amount of from 10 mg to 300 mg, optionally from 90 mg to 270 mg, optionally from 144 mg to 216 mg; and (vi) the green tea extract is present in an amount of from 15 mg to 60 mg, optionally from 20 mg to 50 mg, optionally from 25 mg to 40 mg, optionally from 25 mg to 35 mg.

Embodiment 37 relates to the combination of Embodiment 36, wherein the combination comprises the triterpenoid comprising ursolic acid, where the ursolic acid is present in an amount of from 1.5 mg to 30 mg, optionally from 7.5 mg to 22.5 mg, optionally from 12 mg to 18 mg per serving of the combination.

Embodiment 38 relates to the combination of any one of Embodiments 28-37, wherein the composition comprises, per serving: (i) the D-mannose in an amount of from 1750 mg to 2250 mg, optionally 1950 mg to 2050 mg; (ii) the 2-FL in an amount of from 100 mg to 200 mg, optionally from 140 mg to 160 mg; (iii) the A-type proanthocyanidins in an amount of from 10 mg to 50 mg, optionally from 15 mg to 45 mg; (iv) the quercetin in an amount of from 50 mg to 200 mg, optionally from 50 mg to 150 mg, optionally from 75 mg to 125 mg; (v) the ascorbic acid in an amount of from 100 mg to 260 mg, optionally from 150 mg to 210 mg; optionally, (vi) the green tea extract in an amount of from 15 mg to 45 mg, optionally from 20 mg to 40 mg; and optionally, (vi) the ursolic acid in an amount of from 10 mg to 20 mg, optionally from 12.5 mg to 17.5 mg.

Embodiment 39 relates to the combination of any one of Embodiments 28-38, wherein the combination comprises ursolic acid derived from an extract of sage leaf.

Embodiment 40 relates to the combination of any one of Embodiments 28-39, wherein a single serving of the combination is adapted to be consumed by a human selected from the group of adults, juveniles, and a combination thereof, and wherein the serving is not adapted to be consumed by an infant.

Embodiment 41 relates to a pharmaceutical or nutritional composition comprising the composition of any one of Embodiments 18-27.

Embodiment 42 relates to a pharmaceutical or nutritional composition comprising the combination of any one of Embodiments 28-40.

Embodiment 43 relates to a method of treating, ameliorating, or preventing an infection in a urinary tract of a mammal, the method comprising administering to the mammal the combination of any one of Embodiments 28-40.

Embodiment 44 relates to a method of treating, ameliorating, or preventing an infection in a urinary tract of a mammal, the method comprising administering to the mammal the composition of any one of Embodiments 18-27.

Embodiment 45 relates to a method of treating, ameliorating, or preventing an infection in a urinary tract of a mammal, the method comprising administering to the mammal the pharmaceutical or nutritional composition of Embodiment 41 or 42.

With regard to the description herein, where numerical ranges or limitations are expressly stated, such express ranges or limitations should be understood to include iterative ranges or limitations of like magnitude falling within the expressly stated ranges or limitations (e.g. from about 1 to about 10 includes, 2, 3, 4, etc.; greater than 0.10 includes 0.11, 0.12, 0.13, etc.). For example, whenever a numerical range with a lower limit and an upper limit, is disclosed, any number falling within the range is specifically disclosed. In particular, the following numbers within the range are specifically disclosed: for example, when a variable ranges from 1 percent to 100 percent, this disclosure explicitly includes 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any ratio, expressed as a range defined by two numbers as each number, and variations between listed whole numbers is also specifically disclosed. Use of the term “optionally” with respect to any element of the embodiment, whether included in a claim or not means that the element is required, or alternatively, the element is not required, both alternatives being within the scope of the invention. Use of broader terms such as comprises, includes, and having should be understood to provide support for narrower terms such as consisting of, consisting essentially of, and comprised substantially of.

Additionally, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Likewise, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

Lastly, it is to be understood that the appended claims are not limited to express and particular compounds, compositions, or methods described in the detailed description, which may vary between particular embodiments which fall within the scope of the appended claims. As such, with respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of embodiments and independent and dependent claims, including those related to or dependent on single and/or multiple embodiments and claims, are herein expressly contemplated.

1. A composition comprising:
a monosaccharide comprising a D-mannose;
an oligosaccharide comprising an isolated human milk oligosaccharide (HMO);
a flavonoid comprising an A-type proanthocyanidin;
quercetin;
a vitamin comprising ascorbic acid; and
a green tea extract.

2. The composition of claim 1, wherein: (i) the D-mannose is present in the monosaccharide in an amount of at least 50 wt. %, optionally at least 75 wt. %, or optionally at least 90 wt. %, based on a total weight of the monosaccharide; (ii) the monosaccharide comprising the D-mannose is present in the composition in an amount of from 100 mg to 4,000 mg, optionally from 1,000 mg to 3,000 mg, or optionally from 1,600 mg to 2,400 mg based on a serving of the composition adapted to be consumed by a human selected from the group of adults, juveniles, and a combination thereof, wherein the serving is not adapted to be consumed by an infant; or (iii) both (i) and (ii).

3. The composition of claim 1, wherein: (i) the isolated HMO is present in the oligosaccharide in an amount of at least 50 wt. %, optionally at least 75 wt. %, or optionally at least 90 wt. %, based on a total weight of the oligosaccharide; (ii) the oligosaccharide comprising the isolated HMO is present in the composition in an amount of from 100 mg to 1,000 mg, optionally from 100 mg to 400 mg, optionally from 125 mg to 400 mg, optionally from 125 mg to 300 mg, or optionally from 150 mg to 300 mg based on a serving of the composition adapted to be consumed by a human selected from the group of adults, juveniles, and a combination thereof, wherein the serving is not adapted to be consumed by an infant; (iii) the isolated HMO is selected from the group of 2′-O-fucosyllactose (2′-FL), 3-fucosyllactose (3-FL), 3′-O-sialyllactose (3′-SL), 6′-O-sialyllactose (6′-SL), lacto-N-tetraose (LNT), lacto-N-neotetraose (LNnT), lacto-N-hexaose (LNH), iso-lacto-N-octaose, iso-lacto-N-neooctaose, para-lacto-N-octaose, lacto-N-fucopentaose I (LNFP I), lacto-N-fucopentaose II (LNFP II), lacto-N-fucopentaose III (LNFP III), lacto-N-fucopentaose V (LNFP V), LS-tetrasaccharide a (LST a), LS-tetrasaccharide b (LST b), LS-tetrasaccharide c (LST c), disialyliacto-N-tetraose (DSLNT), difucosyllactose (DFL), and combinations thereof; or (iv) any combination of (i)-(iii).

4. The composition of claim 1, wherein: (i) the A-type proanthocyanidin is present in the flavonoid comprising the A-type proanthocyanidin in an amount of at least 50 wt. %, optionally at least 75 wt. %, or optionally at least 90 wt. %, based on a total weight of the flavonoid comprising the A-type proanthocyanidin; (ii) the flavonoid component comprising the A-type proanthocyanidins is present in an amount of from 1 mg to 100 mg, alternatively from 10 mg to 90 mg, alternatively from 18 mg to 43 mg, alternatively from 18 mg to 36 mg based on a serving of the composition adapted to be consumed by a human selected from the group of adults, juveniles, and a combination thereof, wherein the serving is not adapted to be consumed by an infant; (iii) the flavonoid comprising the A-type proanthocyanidin is derived from an extract of cranberry; or (iv) any combination of (i)-(iii).

5. The composition of claim 1, wherein quercetin is present in the composition in an amount of from 40 mg to 4,000 mg, optionally from 45 mg to 3,000 mg, optionally from 50 mg to 2,400 mg, or optionally from 50 mg to 150 mg based on a serving of the composition adapted to be consumed by a human selected from the group of adults, juveniles, and a combination thereof, wherein the serving is not adapted to be consumed by an infant.

6. The composition of claim 1, wherein: (i) the ascorbic acid is present in the vitamin in an amount of at least 50 wt. %, optionally at least 75 wt. %, or optionally at least 90 wt. %, based on a total weight of the vitamin; (ii) the vitamin comprising ascorbic acid is present in the composition in an amount of from 10 mg to 300 mg, optionally from 90 mg to 270 mg, or optionally from 144 mg to 216 mg based on a total weight of a serving of the composition adapted to be consumed by a human selected from the group of adults, juveniles, and a combination thereof, and wherein the serving is not adapted to be consumed by an infant; or (iii) both (i) and (ii).

7. The composition of claim 1, wherein: (i) the green tea extract is a 6-10/1 extract; (ii) the green tea extract is present in the composition in an amount of from 15 mg to 60 mg, optionally from 20 mg to 50 mg, optionally from 25 mg to 40 mg, or optionally from 25 mg to 35 mg based on a total weight of a serving of the composition adapted to be consumed by a human selected from the group of adults, juveniles, and a combination thereof, and wherein the serving is not adapted to be consumed by an infant; or (iii) both (i) and (ii).

8. The composition of claim 1, further comprising a triterpenoid comprising ursolic acid, wherein: (i) the triterpenoid comprising ursolic acid is derived from an extract of sage leaf; (ii) the ursolic acid is present in the triterpenoid in an amount of at least 50 wt. %, optionally at least 75 wt. %, or optionally at least 90 wt. %, based on a total weight of the triterpenoid; (iii) the triterpenoid comprising ursolic acid is present in an amount of from 1.5 mg to 30 mg, optionally from 7.5 mg to 22.5 mg, or optionally from 12 mg to 18 mg, based on a serving of the composition adapted to be consumed by a human selected from the group of adults, juveniles, and a combination thereof, and wherein the serving is not adapted to be consumed by an infant; or (iv) any combination of (i)-(iii).

9. The composition of claim 1, wherein a serving of the composition comprises a total weight of from 250 mg to 10,000 mg, optionally from 500 mg to 7,500 mg, optionally from 1000 mg to 7,000 mg, or optionally from 2,500 mg to 5,000 mg and is adapted to be consumed by a human selected from the group of adults, juveniles, and a combination thereof, and wherein the serving is not adapted to be consumed by an infant.

10. A combination for use in a method of treating, ameliorating, or preventing an infection in a mammalian urinary tract, the combination comprising the composition of claim 1.

11. The combination of claim 10, wherein:
(i) the D-mannose is present in an amount of from 100 mg to 4,000 mg, optionally from 1,000 mg to 3,000 mg, or optionally from 1,600 mg to 2,400 mg;
(ii) the HMO is present in an amount of from 100 mg to 1,000 mg, optionally from 100 mg to 400 mg, or optionally from 150 mg to 300 mg;
(iii) the A-type proanthocyanidin is present in an amount of from 10 mg to 100 mg, optionally from 15 mg to 90 mg, or optionally from 18 mg to 43 mg;
(iv) the quercetin is present in an amount of from 40 mg to 4,000 mg, optionally from 45 mg to 3,000 mg, or optionally from 50 mg to 2,400 mg;
(v) the ascorbic acid is present in an amount of from 10 mg to 300 mg, optionally from 90 mg to 270 mg, or optionally from 144 mg to 216 mg;
(vi) the green tea extract is present in an amount of from 15 mg to 60 mg, optionally from 20 mg to 50 mg, optionally from 25 mg to 40 mg, or optionally from 25 mg to 35 mg;
(vii) the combination comprises the triterpenoid comprising ursolic acid, where the ursolic acid is present in an amount of from 1.5 mg to 30 mg, optionally from 7.5 mg to 22.5 mg, or optionally from 12 mg to 18 mg;
(viii) any combination of (i) to (vi); or
(ix) any combination of (i) to (vii);
wherein each of the amounts is based on a single serving of the combination adapted to be consumed by a human selected from the group of adults, juveniles, and a combination thereof, wherein the serving is not adapted to be consumed by an infant.

12. The combination of claim 11, comprising, per serving:
(i) the D-mannose in an amount of from 1750 mg to 2250 mg, or optionally 1950 mg to 2050 mg;
(ii) the HMO in an amount of from 100 mg to 200 mg, or optionally from 140 mg to 160 mg;
(iii) the A-type proanthocyanidins in an amount of from 10 mg to 50 mg, or optionally from 15 mg to 45 mg;
(iv) the quercetin in an amount of from 50 mg to 200 mg, optionally from 50 mg to 150 mg, or optionally from 75 mg to 125 mg; and
(v) the ascorbic acid in an amount of from 100 mg to 260 mg, or optionally from 150 mg to 210 mg;
optionally, (vi) the green tea extract in an amount of from 15 mg to 45 mg, or optionally from 20 mg to 40 mg; and
optionally, (vi) the ursolic acid in an amount of from 10 mg to 20 mg, or optionally from 12.5 mg to 17.5 mg;
wherein the serving is adapted to be consumed by a human selected from the group of adults, juveniles, and a combination thereof, wherein the serving is not adapted to be consumed by an infant.

13. The combination of claim 10, comprising ursolic acid derived from an extract of sage leaf.

14. A pharmaceutical or nutritional composition comprising: (i) the composition of claim 1.

15. A method of treating, ameliorating, or preventing an infection in a urinary tract of a mammal, the method comprising administering to the mammal: (i) the composition of claim 1.
